(19) United States
(12) Patent Application Publication
(10) Pub. No.: US 2024/0325783 A1
(43) Pub. Date: Oct. 3, 2024

(54) RADIATION TREATMENT PLAN OPTIMIZATION PER A GENERALIZED METRIC TYPE

(71) Applicant: Siemens Healthineers International AG, Steinhausen (CH)

(72) Inventor: Esa Kuusela, Espoo (FI)

(21) Appl. No.: 18/192,039

(22) Filed: Mar. 29, 2023

(51) Int. Cl. A61N 5/10

(52) U.S. Cl. CPC A61N 5/1031; A61N 5/1048; A61N 5/1081

(57) ABSTRACT

A control circuit accesses information for a given patient (for example, image information corresponding to a target volume and/or an organ-at-risk) as well as characterizing parameters for a given radiation treatment platform (for example, gantry angles). The control circuit can then optimize a radiation treatment plan for the given patient using the given radiation treatment platform as a function, at least in part, of the information for the given patient, the characterizing parameters for the given radiation treatment platform, and a generalized metric type comprising generalized Equivalent Uniform Dose (gEUD)-of-extreme to provide an optimized radiation treatment plan.

### TECHNICAL FIELD

These teachings relate generally to treating a patient's planning target volume with energy pursuant to an energy-based treatment plan and more particularly to optimizing an energy-based treatment plan.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

A radiation treatment plan typically comprises specified values for each of a variety of treatment-platform parameters during each of a plurality of sequential fields. Treatment plans for radiation treatment sessions are often automatically generated through a so-called optimization process. As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

In many cases, the optimization process employs a cost function based on dosimetrical metrics (such as mean dose, dose-at-volume, and/or volume-at-dose) and associated goal values (per, for example, corresponding clinical protocols). In practice, the results of such optimization are sometimes viewed as being unacceptable by the supervising clinicians notwithstanding that the resultant plan may, in fact, be useful and efficacious. The foregoing can occur because characterizations of the optimized plan do not look similar to historical cases that inform the clinician's experience. That dissimilar appearance can lead the clinician to disapprove of the plan and to reengage the process in a search for a more familiar result.

For example, while a clinical protocol may specify that a prescription dose level should cover at least 95% of a patient's target volume, that protocol may be silent as regards dosing in the coldest 5% volumes. Nevertheless, a clinician may have expectations, based on their experience, regarding the shape of the dose volume histogram in that coldest region. An optimizer may satisfy the clinical requirements for the 95% region, and may in fact provide an objectively satisfactory result for the remaining 5% region, but when the latter differs from the clinician's experienced-based expectations, the clinician may subjectively view the optimized plan as being unacceptable.

### BRIEF DESCRIPTION OF THE DRAWINGS

The above needs are at least partially met through provision of the radiation treatment plan optimization per a generalized metric type described in the following detailed description, particularly when studied in conjunction with the drawings, wherein:

FIG. 1 comprises a block diagram as configured in accordance with various embodiments of these teachings;

FIG. 2 comprises a flow diagram as configured in accordance with various embodiments of these teachings;

FIG. 3 comprises a graph as configured in accordance with various embodiments of these teachings;

FIG. 4 comprises a graph as configured in accordance with various embodiments of these teachings;

FIG. 5 comprises a graph as configured in accordance with various embodiments of these teachings; and

FIG. 6 comprises a graph as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, pursuant to these various embodiments, a control circuit accesses information for a given patient (for example, image information corresponding to a target volume and/or an organ-at-risk) as well as characterizing parameters for a given radiation treatment platform (for example, gantry angles). The control circuit can then optimize a radiation treatment plan for the given patient using the given radiation treatment platform as a function, at least in part, of the information for the given patient, the characterizing parameters for the given radiation treatment platform, and a generalized metric type comprising generalized Equivalent Uniform Dose (gEUD)-of-extreme to provide an optimized radiation treatment plan.

By one approach, in lieu of the foregoing or in combination therewith, the aforementioned generalized metric type may comprise generalized Equivalent Uniform Dose (gEUD)-of-most-extreme. By another approach, in lieu of the foregoing or in combination therewith, the aforementioned generalized metric type may comprise generalized Equivalent Uniform Dose (gEUD)-of-hottest. And by yet another approach, and again in lieu of the foregoing or in combination therewith, the generalized metric type may comprise generalized Equivalent Uniform Dose (gEUD)-of-coldest.

By one illustrative example in these regard, the generalized metric type comprises:

$${Q}_{gEUD}^{α} = {( \frac{1}{N} {\sum }_{i} {( {D}_{i} )}^{α} )}^{\frac{1}{α}}$$

where Q_{gEUD}¹ represents a mean dose. (Di is the dose at voxel i (where “i” is an index of the dose voxel) and N represents the number of voxels. α, in turn, is the “gEUD-parameter,” in that one can consider the whole equation as a weighted mean. (In fact, α=1 corresponds to the actual mean, while using larger positive (or negative) α's serve to gradually introduce more emphasis for the hot (or cold) values in the mean.)

By one approach, these teachings will accommodate presenting a display of at least one dose volume histogram that corresponds to the optimized radiation treatment plan.

And, by one approach, these teachings will further accommodate administering therapeutic radiation to the given patient using the given radiation treatment platform as a function of the optimized radiation treatment plan.

So configured, these teachings can provide for improved control of the type of desired dose distributions, at least in part by allowing tuning of the volume upon which the metric is based and also because of how different voxels within that volume are contributing to the metric. For example, the coldest 5% region of a target dose volume histogram can be controlled by setting the goals to a quantity Q_{<}^{5,α} where α is set so that a desired round shape of the dose volume histogram is achieved. For example, α=2 would mean that especially the coldest voxels will be emphasized by the optimizer.

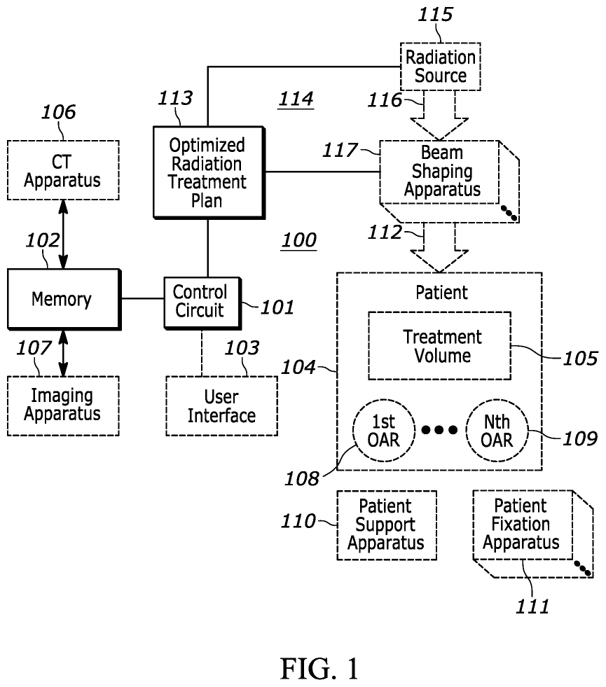

These and other benefits may become clearer upon making a thorough review and study of the following detailed description. Referring now to the drawings, and in particular to FIG. 1, an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101).

In addition to information such as information for a given patient and characterizing parameters for a particular radiation treatment platform as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

By one optional approach the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless platforms, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-based treatment plan (such as, for example, an optimized radiation treatment plan 113). This energy-based treatment plan typically comprises specified values for each of a variety of treatment-platform parameters during each of a plurality of sequential exposure fields. In this case the energy-based treatment plan is generated through an optimization process, examples of which are provided further herein.

By one approach the control circuit 101 can operably couple to an energy-based treatment platform 114 that is configured to deliver therapeutic energy 112 to a corresponding patient 104 having at least one treatment volume 105 and also one or more organs-at-risk (represented in FIG. 1 by a first through an Nth organ-at-risk 108 and 109) in accordance with the optimized energy-based treatment plan 113. These teachings are generally applicable for use with any of a wide variety of energy-based treatment platforms/apparatuses. In a typical application setting the energy-based treatment platform 114 will include an energy source such as a radiation source 115 of ionizing radiation 116.

By one approach this radiation source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the radiation source 115 along that arcuate pathway, and may accordingly control when the radiation source 115 starts moving, stops moving, accelerates, de-accelerates, and/or a velocity at which the radiation source 115 travels along the arcuate pathway.

As one illustrative example, the radiation source 115 can comprise, for example, a radio-frequency (RF) linear particle accelerator-based (linac-based) x-ray source. A linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons.

A typical energy-based treatment platform 114 may also include one or more support apparatuses 110 (such as a couch) to support the patient 104 during the treatment session, one or more patient fixation apparatuses 111, a gantry or other movable mechanism to permit selective movement of the radiation source 115, and one or more energy-shaping apparatuses (for example, beam-shaping apparatuses 117 such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

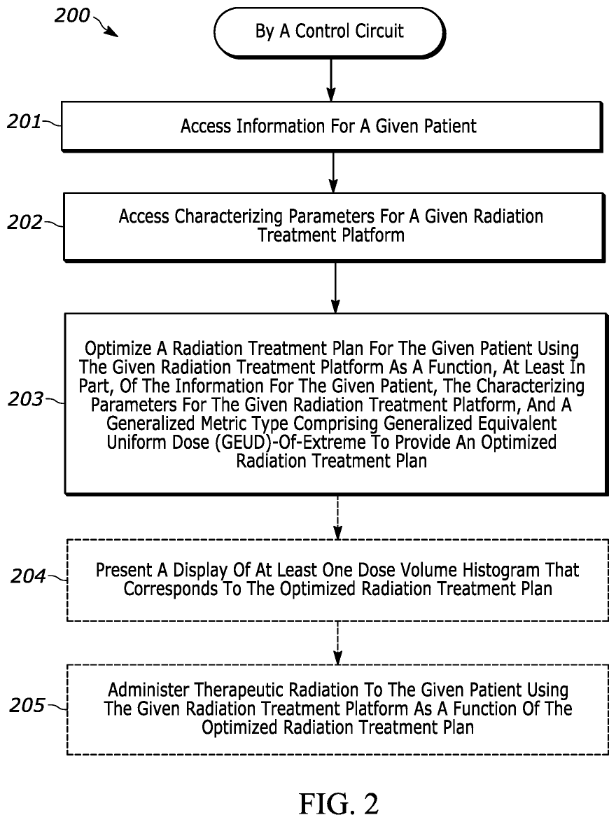

Referring now to FIG. 2, a process 200 that can be carried out, for example, in conjunction with the above-described application setting (and more particularly via the aforementioned control circuit 101) will be described. Generally speaking, this process 200 serves to facilitate generating an optimized radiation treatment plan 113 to thereby facilitate treating a particular patient with therapeutic radiation using a particular radiation treatment platform per that optimized radiation treatment plan.

At block 201, the control circuit 101 accesses information for a given patient. That information might be stored, for example, in the aforementioned memory 102. That information for the given patient may include, at least in part, image information corresponding to at least one of a target volume and an organ-at-risk. In many cases, the patient information will include image information for both a target volume (or volumes) and one or more organs-at-risk.

At block 202, the control circuit 101 accesses characterizing parameters for a given radiation treatment platform. These teachings will accommodate a wide variety of such parameters. In many typical application settings, the accessed characterizing parameters will include a plurality of gantry angles.

Such patient information and such characterizing parameters are well understood in the art. Accordingly, no further elaboration in these regards is provided here for the sake of brevity.

At block 203, the control circuit 101 optimizes a radiation treatment plan for the given patient using the given radiation treatment platform as a function, at least in part, of the information for the given patient, the characterizing parameters for the given radiation treatment platform, and a generalized metric type comprising generalized Equivalent Uniform Dose (gEUD)-of-extreme to provide an optimized radiation treatment plan 113.

As described herein, the generalized metric type is numerically well behaving (and often even convex). By one approach, the generalized metric type represents a combination of two metrics types: (1) the generalized-equivalent-uniform-dose (gEUD) and (2) mean-of-hottest (or coldest). gEUD can be defined as:

$${Q}_{gEUD}^{α} = {( \frac{1}{N} {\sum }_{i} {( {D}_{i} )}^{α} )}^{\frac{1}{α}} ,$$

where Q_{gEUD}¹ is equivalent to the mean dose.

The mean-of-hottest (or -coldest) is another generalization of the mean dose. It can be calculated with the formula:

$${Q}_{>}^{x} = \frac{{\sum }_{i} {D}_{i} θ ( {D}_{i} - {D}_{x \%} )}{{\sum }_{i} θ ( {D}_{i} - {D}_{x \%} )} ( or {Q}_{<}^{x} = \frac{{\sum }_{i} {D}_{i} θ ( {D}_{( 100 - x ) \%} - {D}_{i} )}{{\sum }_{i} θ ( {D}_{( 100 - x ) \%} - {D}_{i} )} ) .$$

θ is a step function (the value of the function is zero if the argument is negative, and 1 if the argument is positive (or zero)). It will be noted that setting x to 100% in either of these formulas will again lead to the mean dose.

Generally speaking, when the metric values are optimized the emphasis is to reduce (or increase) dose in those voxels where the gradient strength is largest. As well, a corresponding gradient strength of zero means that the metric is typically entirely insensitive to those dose levels.

Accordingly, by one approach, the generalized metric type is gEUD-of-hottest (or -coldest). This may be expressed as:

$${Q}_{>}^{x , α} = {( \frac{{\sum }_{i} {( {D}_{i} )}^{α} θ ( {( {D}_{i} )}^{α} - {D}_{x \%} )}{{\sum }_{i} θ ( {( {D}_{i} )}^{α} - {D}_{x \%} )} )}^{\frac{1}{α}} ( or {Q}_{<}^{x , α} = {( \frac{{\sum }_{i} {( {D}_{i} )}^{α} θ ( {D}_{( 100 - x ) \%} - {( {D}_{i} )}^{α} )}{{\sum }_{i} θ ( {D}_{( 100 - x ) \%} - {( {D}_{i} )}^{α} )} )}^{\frac{1}{α}} ) .$$

This approach appropriately tunes the behavior of the objective by using two parameters x and α. The x parameter allows optimization to strictly restrict the effect of the metric into a certain dose region, while the a parameter allows optimization to have uneven emphasize within that region.

By one approach, the generalized metric type can be modified to comprise generalized Equivalent Uniform Dose (gEUD)-of-most-extreme (as distinguished from, for example, a value that is considered extreme but not the most extreme that might be possible in context).

The behavior of the gEUD-of-hottest (or coldest) generalized metric type is potentially similar to some existing metrics in the sense that its value can depend on the doses in a significant number of voxels, such that a small change in any of these point doses may also affect the metrics. The above-described gEUD-of-hottest (or coldest) generalized metric type, however, offers more control over how the changes affect the metric value.

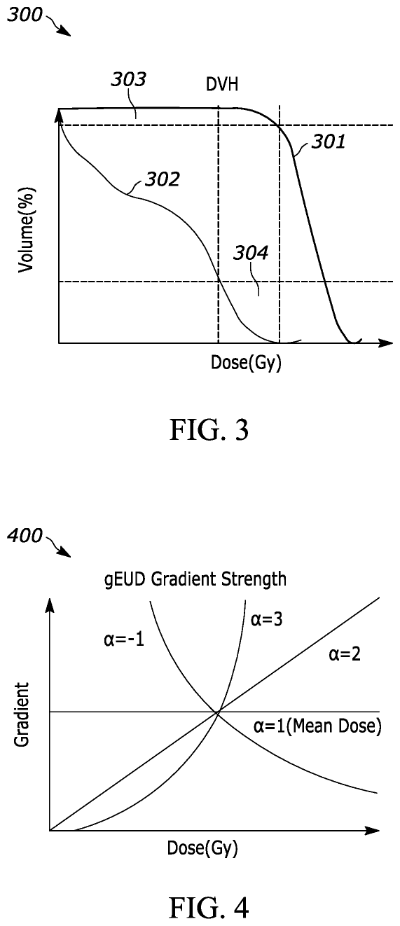

Referring momentarily to FIGS. 3-6, some additional details and insights will be provided. It will be understood that this content is intended to serve an illustrative purpose and is not intended to suggest any limitations as regards the scope of these teachings.

FIG. 3 presents a dose volume histogram plot 300 of a target structure denoted by reference numeral 301 and of an organ-at-risk as denoted by reference numeral 302.

dose volume histograms typically represent three-dimensional dose distributions in a graphical two-dimensional format (the three-dimensional dose distributions being created, for example, in a computerized radiation-treatment planning system based on a three-dimensional reconstruction of an X-ray computed tomography scan and study). The “volume” referred to in dose volume histogram analysis can be, for example, the radiation-treatment target, a healthy organ located near such a target, an arbitrary structure, and so forth.

dose volume histograms are often visualized in either of two ways: as differential dose volume histograms or as cumulative dose volume histograms. With differential dose volume histograms, column height for a given dose bin corresponds to the volume of the structure that receives that dose. Bin doses typically extend along the horizontal axis while structure volumes (either percent or absolute volumes) extend along the vertical axis.

A cumulative dose volume histogram is typically plotted with bin doses along the horizontal axis but has a column height for the first bin that represents the volume of structure(s) that receive greater than or equal to that dose. The column height of the second bin then represents the volume of structure(s) that receive greater than or equal to that dose, and so forth. With high granularity a cumulative dose volume histogram often appears as a smooth line graph. For many application settings cumulative dose volume histograms are preferred over differential dose volume histograms but these teachings can accommodate either approach.

For the sake of an illustrative example, it will be presumed here that there is a need to control either the organ-at-risk dose volume histogram shape in the hottest 30% or the coldest 10% of the target dose volume histogram shape to get the dose volume histogram shapes to appear similar to historical results.

FIG. 4 presents a graph 400 presenting gEUD gradient strength. The gEUD allows the optimizer to focus more on the hottest pixels (or coldest pixels) by adjusting the alpha parameter. While a mean dose metric is affected equally by all voxel doses (and thus the pressure to reduce or increase the point doses is constant), using α>1, (or α<−1) allows the optimizer to focus more on the hottest (coldest) voxels. That said, it can nevertheless be difficult to restrict the behavior to a certain volume size (such as only the coldest 10% of voxels).

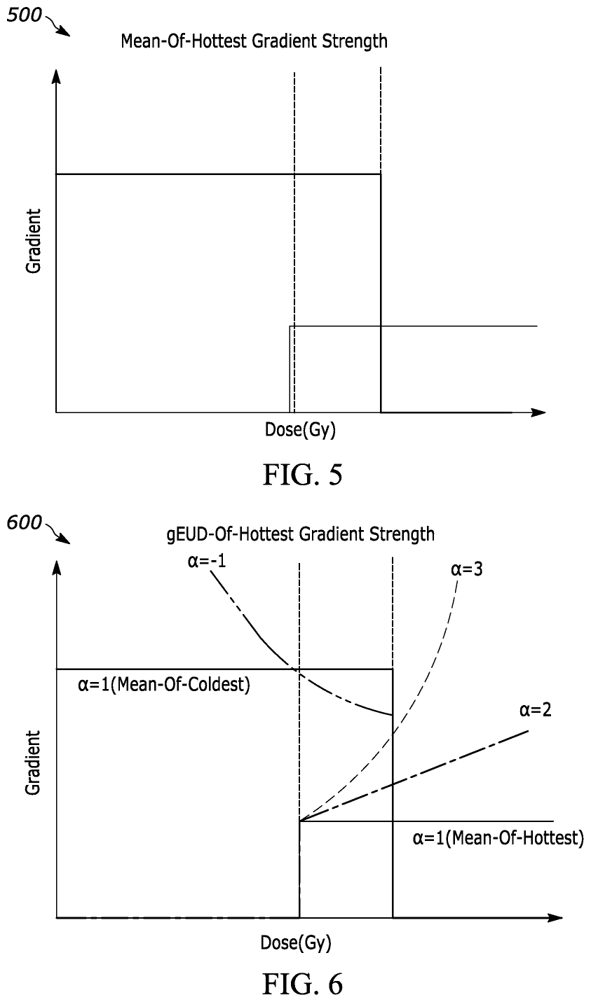

FIG. 5, in turn, presents a graph 500 depicting mean-of-hottest gradient strength. The mean-of-hottest (or -coldest) serves to limit the optimizer to focus exactly to the given volume, but within that volume there is no emphasis as between the hottest and coldest voxels.

FIG. 6 presents a graph 600 depicting gEUD-of-hottest gradient strength in accordance with the present teachings. It can be seen that this metric allows better control of the desired dose distributions. In particular, at the same time as this approach allows tuning of the volume upon which the metric is based, this approach also accommodates how different voxels are contributing to the metric within that volume. For example, the cold 5% region of target dose volume histogram can be controlled by setting the goals to a quantity Q_{<}^{5,α}, where α is set so that a desired round shape of the dose volume histogram is achieved (for example, α=2 would mean that especially the coldest voxels are being emphasized by the optimizer).

With continued reference to the foregoing, and as a further illustrative example, presume that both the target volume dose volume histogram and the organ-at-risk histogram have a region where the user wishes to give guidance related to the desired dose distribution. By way of example, and referring specifically to FIG. 3, for the target volume it can be the cold region (that is, the top-left portion denoted by reference numeral 303), and for the organ-at-risk it can be the high dose tail (that is, the bottom-right quadrant denoted by reference numeral 304).

When using gEUD's as in FIG. 4, The user needs to select the alpha-parameter so that the objective emphasizes either the cold or hot region with a desired degree (for the target volume the choice would perhaps be α<<1, and for the organ-at-risk α>>1), but there is no way to restrict the objective entirely to the hot region.

When mean-of-hottest (or mean-of-coldest) is used as in FIG. 5, one can specify the volume exactly, but within that volume the push to increase (or decrease) the dose is constant. This can lead to possibly a small cold or hot tail.

When the gEUD-of-hottest (or gEUD-of-coldest) is used as in FIG. 6, the user can choose both the desired volume and an a-parameter to control both the volume of influence exactly and also how different dose levels may be weighted within that volume.

So configured, automated radiation treatment planning can be carried out that will yield a plan that is both efficacious in practice and comfortable and familiar to the technician. Any number of benefits are attained by these teachings. For one, a user may more quickly approve a generated optimized radiation treatment plan without needing to repeat the process multiple times to try and achieve a result that feels familiar and experientially comfortable.

With continued reference to FIG. 2, at optional block 204 the control circuit 101 can present a display (via, for example, the aforementioned user interface 103) of at least one dose volume histogram that corresponds to the optimized radiation treatment plan. In this way, the technician conducting the optimization process can view and confirm the usability of the optimized plan in a familiar and appropriate way.

And at optional block 205, these teachings will accommodate administering therapeutic radiation to the given patient using the given radiation treatment platform as a function of that optimized radiation treatment plan.

Those skilled in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

What is claimed is:

1. A method comprising:
by a control circuit:
accessing information for a given patient;
accessing characterizing parameters for a given radiation treatment platform;
optimizing a radiation treatment plan for the given patient using the given radiation treatment platform as a function, at least in part, of the information for the given patient, the characterizing parameters for the given radiation treatment platform, and a generalized metric type comprising generalized Equivalent Uniform Dose (gEUD)-of-extreme to provide an optimized radiation treatment plan.

2. The method of claim 1 wherein the information for the given patient includes, at least in part, image information corresponding to at least one of a target volume and an organ-at-risk.

3. The method of claim 1 wherein the characterizing parameters include a plurality of gantry angles.

4. The method of claim 1 wherein optimizing the radiation treatment plan for the given patient using the given radiation treatment platform as a function, at least in part, of the generalized metric type comprising generalized Equivalent Uniform Dose (gEUD)-of-extreme comprises optimizing the radiation treatment plan for the given patient using the given radiation treatment platform as a function, at least in part, of a generalized metric type comprising generalized Equivalent Uniform Dose (gEUD)-of-most-extreme.

5. The method of claim 1 wherein optimizing the radiation treatment plan for the given patient using the given radiation treatment platform as a function, at least in part, of the generalized metric type comprising generalized Equivalent Uniform Dose (gEUD)-of-extreme comprises optimizing the radiation treatment plan for the given patient using the given radiation treatment platform as a function, at least in part, of a generalized metric type comprising generalized Equivalent Uniform Dose (gEUD)-of-hottest.

6. The method of claim 1 wherein optimizing the radiation treatment plan for the given patient using the given radiation treatment platform as a function, at least in part, of the generalized metric type comprising generalized Equivalent Uniform Dose (gEUD)-of-extreme comprises optimizing the radiation treatment plan for the given patient using the given radiation treatment platform as a function, at least in part, of a generalized metric type comprising generalized Equivalent Uniform Dose (gEUD)-of-coldest.

7. The method of claim 1 further comprising:
presenting a display of at least one dose volume histogram that corresponds to the optimized radiation treatment plan.

8. The method of claim 1 wherein the generalized metric type comprises:
$${Q}_{gEUD}^{α} = {( \frac{1}{N} {\sum }_{i} {( {D}_{i} )}^{α} )}^{\frac{1}{α}}$$
where Q_{gEUD}¹ represents a mean dose.

9. The method of claim 1 further comprising:
administering therapeutic radiation to the given patient using the given radiation treatment platform as a function of the optimized radiation treatment plan.

10. An apparatus comprising:
a memory having stored therein information for a given patient and characterizing parameters for a given radiation treatment platform;
a control circuit operably coupled to the memory and configured to:
access the memory to access the information for the given patient and the characterizing parameters for the given radiation treatment platform;
optimize a radiation treatment plan for the given patient using the given radiation treatment platform as a function, at least in part, of the information for the given patient, the characterizing parameters for the given radiation treatment platform, and a generalized metric type comprising generalized Equivalent Uniform Dose (gEUD)-of-extreme to provide an optimized radiation treatment plan.

11. The apparatus of claim 10 wherein the information for the given patient includes, at least in part, image information corresponding to at least one of a target volume and an organ-at-risk.

12. The apparatus of claim 10 wherein the characterizing parameters include a plurality of gantry angles.

13. The apparatus of claim 10 wherein the control circuit is configured to optimize the radiation treatment plan for the given patient using the given radiation treatment platform as a function, at least in part, of the generalized metric type comprising generalized Equivalent Uniform Dose (gEUD)-of-extreme by optimizing the radiation treatment plan for the given patient using the given radiation treatment platform as a function, at least in part, of a generalized metric type comprising generalized Equivalent Uniform Dose (gEUD)-of-most-extreme.

14. The apparatus of claim 10 wherein the control circuit is configured to optimize the radiation treatment plan for the given patient using the given radiation treatment platform as a function, at least in part, of the generalized metric type comprising generalized Equivalent Uniform Dose (gEUD)-of-extreme by optimizing the radiation treatment plan for the given patient using the given radiation treatment platform as a function, at least in part, of a generalized metric type comprising generalized Equivalent Uniform Dose (gEUD)-of-hottest.

15. The apparatus of claim 10 wherein the control circuit is configured to optimize the radiation treatment plan for the given patient using the given radiation treatment platform as a function, at least in part, of the generalized metric type comprising generalized Equivalent Uniform Dose (gEUD)-of-extreme by optimizing the radiation treatment plan for the given patient using the given radiation treatment platform as a function, at least in part, of a generalized metric type comprising generalized Equivalent Uniform Dose (gEUD)-of-coldest.

16. The apparatus of claim 10 wherein the control circuit is further configured to:
present a display of at least one dose volume histogram that corresponds to the optimized radiation treatment plan.

17. The apparatus of claim 10 wherein the generalized metric type comprises:
$${Q}_{gEUD}^{α} = {( \frac{1}{N} {\sum }_{i} {( {D}_{i} )}^{α} )}^{\frac{1}{α}}$$
where Q_{gEUD}¹ represents a mean dose.

18. The apparatus of claim 10 wherein the control circuit is further configured to:
facilitate administering therapeutic radiation to the given patient using the given radiation treatment platform as a function of the optimized radiation treatment plan.
